(19) United States
(12) Patent Application Publication
(10) Pub. No.: US 2010/0028413 A1
(43) Pub. Date: Feb. 4, 2010

(54) SKIN TREATMENT STRIP

(76) Inventor: Aida Picone, Woodland Hills, CA (US)

Correspondence Address: KELLY LOWRY & KELLEY, LLP, 6320 CANOGA AVENUE, SUITE 1650, WOODLAND HILLS, CA, 91367, US

(21) Appl. No.: 12/534,512

(22) Filed: Aug. 3, 2009

Related U.S. Application Data
(60) Provisional application No. 61/085,924, filed on Aug. 4, 2008.

(51) Int. Cl. A61K 9/70; A61K 33/40; A61P 17/00

(52) U.S. Cl. 424/449; 424/616

(57) ABSTRACT

The skin treatment strip includes a base liner and an applicant liner coupled to the base liner by an adhesive. The skin treatment strip further includes a chemical layer sandwiched between the base liner and the applicant liner that is reactive to a skin blemish. The chemical layer includes a weak acid such as hydrogen peroxide for treating the skin blemish, a base such as sodium hydroxide for balancing skin pH and preventing skin irritation, a sugar-based alcohol such as glycerin for soothing the skin and a water-based carrier. Preferably, the adhesive is used to maintain contact of the chemical layer with the skin blemish upon application of the applicant liner.

### BACKGROUND OF THE INVENTION

The present invention relates to a skin treatment strip. More particularly, the invention relates to a treatment strip having a composition for quickly treating skin conditions without further irritating the skin.

Skin conditions such as acne and other skin blemishes affect a large percentage of the population, including both adolescents and adults. Acne occurs primarily on facial skin and may appear, although to a lesser extent, on other parts of the body. Multiple factors are thought to cause or influence the development of acne and facial blemishes, including particles in the environment, genetics, temperature, diet, drugs, irritants, rapidly changing hormones (especially in adolescents), or emotional stress. For instance, during adolescence, glands and follicles in the skin tend to increase in activity and can leave the skin oily and greasy. These oils naturally protect the skin from disease and infection. But, partial obstruction of the glands or follicles may cause inflammation of the surrounding tissue. As a result, acne forms and may cause red blemishes or scarring in affected areas. Genetic factors may also influence the onset, severity and pattern of outbreaks. Other triggering factors such as changes in air temperature, food, facial makeup and sunlight exposure are often uncontrollable and impracticable or impossible to avoid. Skin conditions often associated with acne and facial blemishes include dryness, dandruff and other redness or noticeable skin irritations.

A number of different treatments have been developed over the years to treat such skin conditions. Treatments include diet regulation, skin care cleansers, local or oral antibiotics, application of acidic substances, and hormone or anti-androgen pills, just to name a few. One or multiple treatments may be used individually or simultaneously. Another, more natural approach includes utilizing juniper extracts to thin oily or greasy skin secretions. Other natural ingredients may include Vitamin A, Aloe Vera or Chamomile extracts. Unfortunately, no single therapeutic remedy has been effective for the majority of all affected individuals. Though some treatments may be effective over time, consumers may not be satisfied with the speed or performance of these formulations. Moreover, the prior art fails to provide an easy and adequate skin treatment product absent using messy creams or undesirable antibiotics.

One particularly popular approach to treat acne and related blemishes involves the application of an acid-based formula to the skin. One problem with moderate to strong acid-based formulas is that effective amounts may cause stinging sensations and skin irritations that cause redness. In some aspects of the prior art, the stinging sensation and redness is considered a sign of effective performance. Other, acidic-based medicines or compositions may cause unacceptable skin bleaching and removal, thereby increasing susceptibility to other skin irritations. Bleaching could also be particularly undesirable for patients that have highly pigmented skin. Alternatively, some products include additional irritation suppressants or moisturizers to counteract the irritations caused by the acid-based medicines. In one aspect, these compositions are able to at least partially prevent further skin blemishes that result from application from the acid-based composition. As an alternative, ammonia salts may be used in place of the aforementioned acid-based formulas as a less intensive acne or blemish treatment.

Another prior art technique to remove blemishes from facial skin is by removing a portion of the epidermis. One exfoliation approach applies an adhesive chemical-base layer to exfoliate the skin. The chemicals interact with the outer layers of the skin. A portion of the epidermis adheres to the product and peels away when the product is removed. Other similar exfoliation approaches utilize items commonly referred to as “face masks” or “beauty masks”. These techniques require applying a viscous adherent material to a portion of the body to be exfoliated. The substance is allowed to dry or “set up”. The dried and solidified material is then peeled off the body simultaneously removing unwanted particles in the epidermis. Another adhesive exfoliation technique is generally known as “tape stripping”. Tape stripping involves applying tape having a pressure-sensitive adhesive backing to the skin. The strip is then pulled away and the adhesive removes an outer layer of the skin, including dirt and other pore clogging materials. Another, less popular and significantly more expensive treatment, includes surgical techniques utilizing a laser that removes layers of the epidermis.

There are certainly many drawbacks associated with the above-identified treatments. Prescription or over-the-counter medications tend to be extremely expensive and may not produce acceptable results. Over time, local antibiotics may cause local or systemic sensitivity to the drug, thereby limiting the future effectiveness of the drug. Antibiotics and other prescriptions or over-the-counter drugs often have a risk of developing other complications or side effects. A user may also experience increased tolerance with regard to oral drugs. In one particular instance, some acne drugs are known to cause systemic side effects and cannot be used during pregnancy. There are also obvious risks associated with treatments based upon manipulating hormonal balances. Consumers, of course, prefer performance without side effects.

Another approach to reduce the visual appearance of skin blemishes is to apply cosmetic-based compositions that cover and hide the epidermis. These compositions are generally well known in the art and endeavor to decrease visual redness and irritation associated with facial blemishes. Products including masking creams, makeup and the like are commonplace. Longer term medical treatment may also be desirable for clearing redness and other, more severe, acne conditions. But, it is often desirable to reduce redness associated with facial blemishes in a relatively short time period. This avoids having to mask blemishes with unsightly coverings that may interfere with the normal application of facial makeup.

There exists therefore a significant need for a skin treatment product that quickly and adequately reduces the visual appearance of skin conditions, such as acne and other blemishes, without undesirable side effects. Such a skin treatment product should include a weak acid for treating the irritated skin, a base to balance skin pH and prevent irritation from the acid, a water-based carrier solution, and sugar-based alcohols to sooth the skin. Moreover, such a skin treatment product should be manufactured in multiple strips or multiple detachable pads, should include a releasable adhesive for applying the pad to a blemish on the skin and should be easily removable from the skin such that the adhesive does not cause further irritation. The skin treatment strip in accordance with the present invention should be easy to manufacture and cost-effective. The present invention fulfills these needs and provides further related advantages.

### SUMMARY OF THE INVENTION

The skin treatment strip disclosed herein includes a base liner and an applicant liner coupled to the base liner by an adhesive. The skin treatment strip further includes a chemical layer sandwiched between the base liner and the applicant liner that is reactive to a skin blemish. The chemical layer itself may include a weak acid for treating the skin blemish, a base for balancing skin pH and preventing skin irritation, a sugar-based alcohol for soothing the skin and a water-based carrier. In a particularly preferred embodiment, the weak acid includes hydrogen peroxide, the sugar-based alcohol includes glycerin, and the base includes sodium hydroxide. In one embodiment, the adhesive is non-removably attached to the base liner. Here, the user peels back the applicant liner for manual application to the skin blemish for a predetermined amount of time. Since the adhesive remains on the base liner, the user must apply constant force to the applicant liner to maintain contact between the chemical layer and the skin blemish. The base liner and the applicant liner may be flexible, and in a particularly preferred embodiment, the applicant liner is substantially more flexible than the base liner to facilitate removal therefrom.

In an alternative embodiment, the adhesive and the chemical layer are homogenously mixed together and disposed between the base liner and the applicant liner. In this embodiment, the adhesive and the chemical layer are non-removably attached to the applicant liner. When removed from the base liner, the exposed adhesive attaches the applicant liner, having the chemical layer thereon, to the skin blemish. After application, the user is not required, as were in the above-described embodiment, to maintain pressure on the applicant liner to ensure constant contact of the chemical layer with the skin blemish. Instead, the adhesive maintains contact of the chemical layer with the skin blemish. Alternatively, the skin treatment strip may include a second adhesive, wherein the first adhesive is disposed around the periphery of the applicant liner and the second adhesive is non-removably attached to the base liner and disposed thereon in a non-overlapping relationship with the first adhesive. In this embodiment, the chemical layer is not mixed with either the first adhesive or the second adhesive. The first adhesive may include a different adhesive than the second adhesive. Moreover, the skin treatment strip may include a plurality of treatment pads that interconnect into an elongated strip. Each pad may be separable from the elongated strip by a score line.

Other features and advantages of the present invention will become apparent from the following more detailed description, when taken in conjunction with the accompanying drawings, which illustrate, by way of example, the principles of the invention.

### BRIEF DESCRIPTION OF THE DRAWINGS

The accompanying drawings illustrate the invention. In such drawings:

FIG. 1 is a perspective view of a skin treatment strip in accordance with the present invention;

FIG. 2 is an enlarged cross-sectional view of the skin treatment strip of FIG. 1, taken along the line 2-2;

FIG. 3 is a perspective view illustrating removing a skin treatment pad from the skin treatment strip;

FIG. 4 illustrates an alternative skin treatment pad torn away from the skin treatment strip;

FIG. 5 is an environmental view of a person with a skin condition;

FIG. 6 is an environmental view illustrating application of the skin treatment pad to the skin condition; and

FIG. 7 is an environmental view illustrating removal of the skin treatment pad from the treated area.

### DETAILED DESCRIPTION OF THE PREFERRED EMBODIMENTS

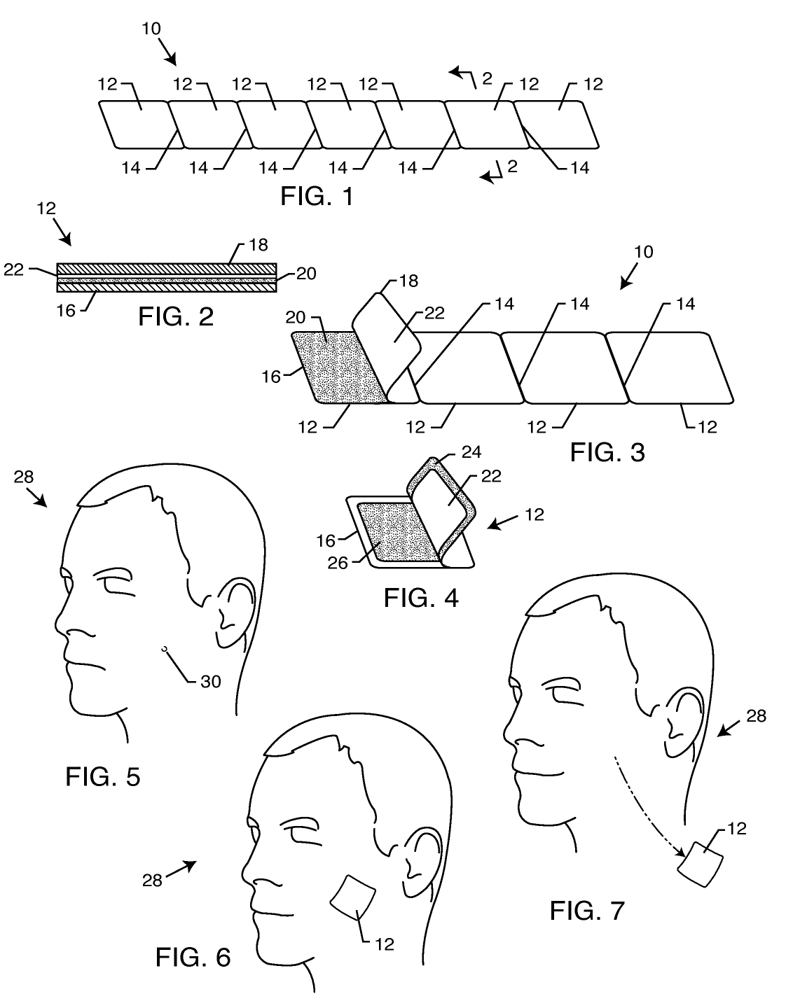

As shown in the drawings for purposes of illustration, the present invention for a skin treatment strip is referred to generally by the reference number 10. In FIG. 1, the skin treatment strip 10 is illustrated having a plurality of skin treatment pads 12, which ultimately comprise the skin treatment strip 10. The skin treatment pads 12 are each interconnected via a set of scorelines 14. The scorelines 14 allow a user to tear each individual skin treatment pad 12 away from the skin treatment strip 10. This allows the skin treatment strip 10 to be manufactured in a way where a user may utilize one portion of the skin treatment strip 10 instead of using the entire skin treatment strip 10 in one application. For larger applications, two or more skin treatment pads 12 may be torn away from the skin treatment strip 10. The entire skin treatment strip 10 could even be used itself. Alternative embodiments include providing the skin treatment strip 10 in sheet form such that the skin treatment pads 12 form multiple rows and columns. The skin treatment pads 12 may also be manufactured individually and be provided as a stack of pads 12. Moreover, each skin treatment pad 12 may include additional scorelines (not shown) wherein a user may tear away portions of the pads 12 depending upon the desired application. The skin treatment strip 10, the skin treatment pads 12 and the scorelines 14 may be arranged and manufactured for general skin application or specially arranged and manufactured for application to a specific portion of the body.

FIG. 2 illustrates a cross-sectional view of the skin treatment pad 12. As shown, the skin treatment pad 12 generally includes a base liner 16 and a top layer 18. Sandwiched between the base liner 16 and the top layer 18 is an adhesive 20 and a chemical layer 22. The base liner 16 is a carrier liner for the top layer 18, the adhesive 20 and the chemical layer 22. Preferably, the base liner 16 is made from a flexible material and is capable of receiving the adhesive 20 thereon. The adhesive 20 may be a dual purpose adhesive such that it 1) retains the chemical layer 22 and/or the top layer 18 to the base liner 16 and; 2) is capable of subsequently adhering the top layer 18 to the skin or other surface. Alternatively, the adhesive 20 may comprise two adhesives. In this embodiment in FIG. 4, a first adhesive 24 is applied around the periphery of the base liner 16 and pulls away with the top layer 18 for application to a treatment location. A second adhesive 26, shown disposed inside of the exterior periphery of the base liner 16 in FIG. 4, preferably adhesively attaches the top layer 18 and the chemical layer 22 to the base liner 16. In this embodiment, the second adhesive 26 remains on the base liner 16 when the top layer 18 is removed therefrom. The first adhesive 24 and the second adhesive 26 may be the same or different types of adhesive.

FIGS. 3 and 4 illustrate partial removal of the skin treatment pad 12. Specifically in FIG. 3, the top layer 18 peels back from the base liner 16. In this embodiment, the base liner 16 retains the adhesive 20 thereon. Accordingly, the top layer 18 retains the chemical layer 20, which may have a non-stick surface. In this case, the skin treatment pad 12 must be held against the affected surface or otherwise attached thereto by any other method known in the art other than by a sticky adhesive (e.g. a carbonate material, a dry-stick adhesive, static cling, etc.). Alternatively, the top layer 18 retains at least a partial mixture of the adhesive 20 and the chemical layer 22 such that the top layer 18 may directly affix to the skin or other related surface. In this embodiment, a person of ordinary skill in the art will readily recognize that the adhesive layer 20 and the chemical layer 22 homogenously form a single layer sandwiched between the base liner 16 and the top layer 18, opposed to the dual layer illustrated in FIG. 2.

For larger applications, multiple top layers 18 of multiple skin treatment pads 12 may be removed from the skin treatment strip 10 simultaneously. Here, the top layers 18 stay connected by the scorelines 14. Alternatively, an individual skin treatment pad 12 (FIG. 4) may be disconnected from the skin treatment strip 10 about the scorelines 14 (FIG. 3). In this embodiment, a single top layer 18 is removed from the individual skin treatment pad 12, as illustrated in FIG. 4. FIG. 4 also illustrates the top layer 18 having the first adhesive 24 disposed around an outer periphery thereof and the second adhesive 26 remaining adhesively fixed to the base liner 16. In this embodiment, the chemical layer 22 is exposed and remains on the top layer 18 as the top layer 18 is peeled back away from the base liner 16. The first adhesive 24 is also exposed and peels away from the base liner 16 with the top layer 18. The first adhesive 24 may adhere the skin treatment pad 12 to the skin or other desired treatment surface. Accordingly, the second adhesive 26 remains on the base liner 16 to be later discarded therewith. In another alternative embodiment, the first adhesive 24 may be disposed on the base liner 16 for removably retaining the adhesive layer 20 thereon and the second adhesive 26 may be disposed between the top layer 18 and the chemical layer 22 for carrying the chemical layer 22 on the top layer 18.

Both the base liner 16 and the top layer 18 are preferably flexible. Flexibility facilitates removability of the top layer 18 from the base liner 16. In this regard, a user may bend the skin treatment strip 10 to access either the base liner 16 or the top layer 18. Individual skin treatment pads 12 may also be bent about the scorelines 14 to weaken the connection therebetween. In one embodiment, the base liner 16 is relatively rigid compared to the top layer 18. A rigid base liner 16 providers a sturdier carrier sheet. But, the top layer 18 is preferably flexible to maximize conformable attachment to the skin or other body part to be treated by the skin treatment pad 12.

FIGS. 5-7 illustrate a set of environmental views of a person 28 having a skin condition 30 (FIG. 5) removed therefrom (FIG. 7) by application of the skin treatment pad 12. Accordingly, the skin condition 30 may encompass any one of a variety of skin irritations that include acne, eczema, dry skin, or other redness or swelling due to blockage or irritation of the epidermal facial tissue. Moreover, the scope of the skin treatment strip 10 extends to a wide variety of blemish-type skin irritations all over the body, including the face. For example, the skin treatment pad 12 is effective at treating skin conditions by removing brown spots and other discolorations of the skin caused by sun damage, lightening hair on the face or other areas of the body, and eliminating dark circles around the eyes. As shown in FIGS. 6 and 7, the skin treatment pad 12 is simply applied to the skin condition 30 for an effective duration to provide substantial healing. In this regard, the skin condition 30 is no longer existent (FIG. 7) upon removal of the skin treatment pad 12. Removal of the skin condition 30 does not require the use of messy creams or the application of antibiotics or other medications. Rather, the active ingredients of the skin treatment pad 12 are applied topically by the chemical layer 22.

The chemical layer 22 preferably comprises a combination of water, glycerin, hydrogen peroxide, a carbomer, sodium hydroxide and sodium saccharin. In a particularly preferred embodiment, the chemical layer 22 comprises the dental whitening formula used in the Crest Whitestrips Classic manufactured by Proctor & Gamble of Cincinnati, Ohio. Accordingly, the water moisturizes and tends to soften the skin such that the other ingredients may actively reduce the visual appearance of the aforementioned skin conditions such as facial blemishes, brown spots from sun damages and dark eye circles. The hydrogen peroxide is a weak acid that is balanced by other basic compounds in the chemical layer 22. Hydrogen peroxide has strong oxidizing properties and is used as a disinfectant (as an oxidizer) and as an antiseptic. Hydrogen peroxide is particularly beneficial for cleaning and removing dead tissue from the epidermis and may also relieve pressure within small vessels underneath the skin. This eases the redness and irritation associated with facial blemishes. Hydrogen peroxide may also be used as an anti-microbial agent to further eliminate infection of the blemish area. Glycerin is an alcohol-based sugar that has a high solubility in the water and has a basic pH that balances out the lightly acidic-based hydrogen peroxide. Notably, glycerin has moisturizing properties and prevents skin dryness. In turn, moisturizing the skin, namely areas of blemishes and other skin conditions, reduces redness and swelling associated thereto. Moisturization also counteracts any further irritation due to application of the acidic compounds. Glycerin may also be used as a light bonding agent to adhesively retain the top layer 18 of the skin treatment pad 12 around the skin condition. Users may also experience a cooling or refreshing sensation from the glycerin when the skin treatment pad 12 is applied to the skin. The sodium hydroxide is a stronger chemical base that helps reduce irritations associated with applying an acid to the skin. The carbomer is a substantially stable alkaline whose chemical makeup depends on the number of alkyne bonds introduced into an alkaline. The carbomer stabilizes and balances the overall organic composition of the chemical layer 22. Lastly, the sodium saccharin is used as a mild acid to further cleanse and remove tissue of the affected area of the skin condition.

The skin treatment pad 12 may be packaged in a variety of products. For instance, the chemical layer 22 may vary depending on the desired effective amounts of the above-identified ingredients. In turn, each skin treatment pad 12 may have a different chemical layer 22 and may therefore require different application durations. For example, a small blemish may require application of a chemical layer 22 formulated for providing superficial cleansing. In this embodiment, the chemical layer 22 is designed to provide cleansing of the epidermis in a matter of minutes. Alternatively, the skin treatment pad 12 may include a chemical layer 22 that is more reactive with the skin to penetrate and heal deeper skin conditions. In this embodiment, the person 28 may need to apply the skin treatment pad 12 for a longer duration or, for example, at multiple intervals throughout the day or across multiple days. Of course, the skin treatment pad 12 should temporarily and lightly adhere to the skin. In this regard, the skin treatment pad 12 should not strongly adhere to the affected area such that removal of the skin treatment pad 12 causes further discomfort or irritation of the epidermis. In an alternative embodiment, the person 28 simply holds the skin treatment pad 12 to the skin condition 30 for a short duration, instead of using an adhesive, such as the first adhesive 24.

Although several embodiments have been described in detail for purposes of illustration, various modifications may be made to each without departing from the scope and spirit of the invention. Accordingly, the invention is not to be limited, except as by the appended claims.

What is claimed is:

1. A skin treatment strip, comprising:
a base liner;
an applicant liner coupled to the base liner by an adhesive; and
a chemical layer sandwiched between the base liner and the applicant liner and reactive to a skin blemish, the chemical layer comprising a weak acid for treating the skin blemish, a base for balancing skin pH and preventing skin irritation, a sugar-based alcohol for soothing the skin, and a water-based carrier.

2. The treatment strip of claim 1, wherein the weak acid comprises hydrogen peroxide, the sugar-based alcohol comprises glycerin, and the base comprises sodium hydroxide.

3. The treatment strip of claim 1, wherein the adhesive and the chemical layer are homogenously mixed together and disposed between the base liner and the applicant liner.

4. The treatment strip of claim 1, wherein the adhesive and the chemical layer are non-removably attached to the applicant liner.

5. The treatment strip of claim 4, wherein, after removal from the base liner, the applicant liner attaches to the skin blemish by the adhesive.

6. The treatment strip of claim 4, including a second adhesive, wherein the first adhesive is disposed around the periphery of the applicant liner and the second adhesive is non-removably attached to the base liner and disposed thereon in a non-overlapping relationship with the first adhesive.

7. The treatment strip of claim 6, wherein the first adhesive comprises a different adhesive than the second adhesive.

8. The treatment strip of claim 1, wherein the skin treatment strip comprises a plurality of treatment pads.

9. The treatment strip of claim 8, wherein the plurality of treatment pads interconnect into an elongated strip and are separable therefrom by a scoreline.

10. The treatment strip of claim 1, wherein the base liner and the applicant liner are flexible.

11. The treatment strip of claim 10, wherein the applicant liner is more flexible than the base liner.

12. The treatment strip of claim 1, wherein the adhesive is non-removably attached to the base liner.

13. A skin treatment strip, comprising:
a base liner;
an applicant liner coupled to the base liner by a first adhesive non-removably disposed around the periphery of the base liner; and
a chemical layer homogenously mixed together with a second adhesive attached to the applicant liner and disposed between the base liner and the applicant liner in a non-overlapping relationship with the first adhesive, the chemical layer reactive to a skin blemish and includes a weak acid comprising hydrogen peroxide for treating the skin blemish, a base comprising sodium hydroxide for balancing skin pH and preventing skin irritation, a sugar-based alcohol comprising glycerin for soothing the skin, and a water-based carrier.

14. The treatment strip of claim 13, wherein the second adhesive and the chemical layer are non-removably attached to the applicant liner such that, after the applicant liner is removed from the base liner, the exposed adhesive attaches the applicant liner to the skin blemish.

15. The treatment strip of claim 13, wherein the first adhesive comprises a different adhesive than the second adhesive.

16. The treatment strip of claim 13, wherein the skin treatment strip comprises a plurality of treatment pads.

17. The treatment strip of claim 16, wherein the plurality of treatment pads interconnect into an elongated strip and are separable therefrom by a scoreline.

18. The treatment strip of claim 13, wherein the applicant liner is more flexible than the base liner.

19. A skin treatment strip, comprising:
a flexible base liner;
an applicant liner relatively more flexible than and coupled to the base liner by an adhesive non-removably disposed around the periphery of the base liner; and a chemical layer homogenously mixed together with a second adhesive non-removably attached to the applicant liner and disposed between the base liner and the applicant liner in a non-overlapping relationship with the first adhesive, the chemical layer reactive to a skin blemish and includes a weak acid comprising hydrogen peroxide for treating the skin blemish, a base comprising sodium hydroxide for balancing skin pH and preventing skin irritation, a sugar-based alcohol comprising glycerin for soothing the skin, and a water-based carrier; wherein when removed from the base liner, the exposed second adhesive attaches the applicant liner to the skin blemish.

20. The treatment strip of claim 1, wherein the skin treatment strip comprises a plurality of treatment pads interconnected in an elongated strip separable by a set of scorelines and the first adhesive is non-removably attached to the base liner and comprises a different adhesive than the second adhesive.
